(19) United States
(12) Patent Application Publication
(10) Pub. No.: US 2009/0054929 A1
(43) Pub. Date: Feb. 26, 2009

(54) Apparatus for the Prevention and Treatment of Back Pain

(76) Inventor: Francisco E. Pajares Mellado, Madrid (ES)

Correspondence Address: Quinn Emanuel Urquhart Oliver & Hedges;Koda/Androlia, 865 S. Figueroa Street, 10th Floor, Los Angeles, CA, 90017, US

(21) Appl. No.: 11/887,853

(22) PCT Filed: Jul. 13, 2006
(86) PCT No.: PCT/ES2006/000401

§ 371 Date: Oct. 3, 2007

(30) Foreign Application Priority Data
Jul. 26, 2005 (ES) U200501715

(51) Int. Cl. A61B 17/70; A61F 5/00

(52) U.S. Cl. 606/246; 602/19

(57) ABSTRACT

The invention relates to an apparatus for the prevention and treatment of back pain, which is based on lumbar, dorsal and cervical strength training machines. The inventive apparatus comprises three vertebral supports which are disposed at a determined angle and which are used to strengthen and stretch (separation of the vertebral joints) the lumbar, dorsal and cervical areas of the back simultaneously in the appropriate movement plane. In order to prevent and treat back pain, once the back has been suitably positioned, all of the back must undergo simultaneous symmetrical strengthening. For said purpose, the apparatus consists of a vertical iron structure comprising three supports, which is perfectly articulated with a horizontal iron structure comprising a seat which can be used to perform seated exercises. According to the invention, the back is pushed against the cervical, dorsal and lumbar supports in order to produce symmetrical strengthening and stretching movements which can be adjusted to suit the individual using height and resistance control means, such as to have a decompressive effect on the vertebral column.

### OBJECT OF THE INVENTION

This invention relates to an apparatus for the prevention and treatment of the back pain caused by numerous pathologies.

### BACKGROUND OF THE INVENTION

It is based on fixed lumbar, dorsal and cervical strength training machines; these training machines only incorporate one of the three supports, therefore they only strengthen in an isolated manner one of the three areas of the back. Besides, with fixed training machines, on carrying out the exercise on only one part of the back, this is carried out on an inappropriate movement plane.

### DESCRIPTION OF THE INVENTION

The improvements proposed by the invention resolve the problems outlined above, improving the prevention and treatment of back pain.

The “Apparatus for the Prevention and Treatment of Back Pain” incorporates three vertebral supports disposed at a determined angle which promote the strengthening and stretching (separation of the vertebral joints) of the lumbar, dorsal and cervical areas of the back at the same time and on an appropriate movement plane.

In order to prevent and treat back pain it is necessary to first place the back in a suitable position, that is to say, with vertebral stretching (separation of the vertebral joints) of all the vertebral column at the same time; in order to do this, this invention comprises three vertical supports with an iron structure, padded with foam-rubber and upholstered in leather. The upper/lower forward/rearward obliquity of the lumbar support achieves separation of the lumbar vertebral joints. The placement of the dorsal support perpendicular to our vertebral column favours the separation of the dorsal vertebral joints. The upper/lower forward/rearward obliquity of the cervical support brings about a head-flexing which achieves the separation of the cervical vertebral joints. The cervical, dorsal and lumbar supports may be adjusted in height on the vertical iron structure, which permits each person to adjust the supports to his/her own back. In order to prevent and to treat back pain it is necessary, once we have managed to accommodate our back in a suitable position, to carry out a symmetrical strengthening of the entire back at the same time; for this reason the “Apparatus for the Prevention and Treatment of Back Pain” features a vertical iron structure with three supports, perfectly articulated with a horizontal iron structure which incorporates a seat which can be used to perform seated exercises. When we press our back against the cervical, dorsal and lumbar supports, this brings about a stretching and a symmetrical strengthening of the back muscles in an individual manner, thanks to its height and resistance control means, achieving a decompressive effect on the vertebral column.

### DESCRIPTION OF THE DRAWINGS

An example for the embodiment of this invention is explained below in greater detail and by means of the attached drawings, in which:

FIG. 1 portrays the side view of the assembly, of the “Apparatus for the Prevention and Treatment of Back Pain” at the initial stage of the exercise.

FIG. 2 portrays the side view of the assembly, of the “Apparatus for the Prevention and Treatment of Back Pain” at the final stage of the exercise.

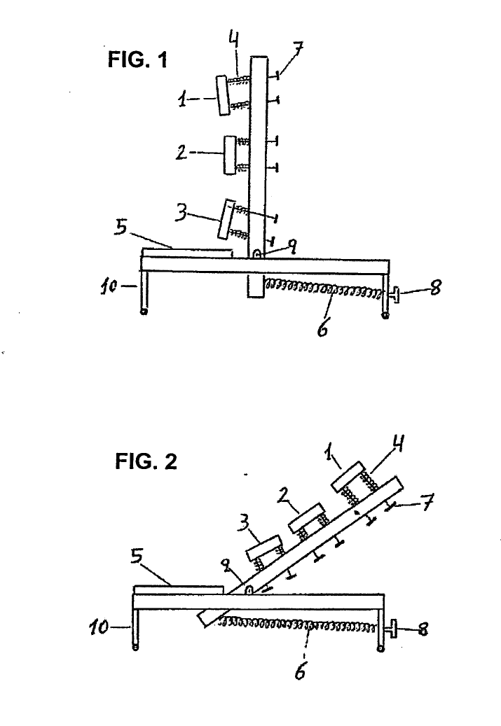

It is comprised of two iron structures articulately joined together. It is to the vertical iron structure that the cervical (1), dorsal (2) and lumbar (3) supports are attached; these supports are arranged at a determined angle and incorporate compression springs 4 which allow a certain amount of movement on the same support plane. The height of the cervical (1), dorsal (2) and lumbar (3) supports may be adjusted by means of the height control means (7) on the vertical iron structure; this allows each person to adjust the supports to suit his/her back. It is on the horizontal iron structure that the seat (5) is located, on which persons may sit; the pivot (9), which is articulated with the vertical iron structure; the legs (10), which raise the structure from the floor, and the traction spring (6), which incorporates a resistance control means (8), which allows each person to adjust the resistance of the “Apparatus for the Prevention and Treatment of Back Pain”.

The materials, shape, size and arrangement of the elements shall be susceptible to variations on condition that this shall not entail a modification of the essential nature of the invention.

1. An apparatus for the prevention and treatment of back pain, consisting of three vertebral supports; one cervical (1), another dorsal (2) and a third lumbar (3), with an upper/lower forward/rearward angulation or obliquity of the lumbar support, the dorsal support being perpendicular to our vertebral column and the upper/lower forward/rearward obliquity of the cervical support achieves a stretching (separation of the vertical joints) of the vertebral column.

2. An apparatus for the prevention and treatment of back pain, according to claim 1, wherein the inclusion of three vertebral supports allows the strengthening of the lumbar, dorsal and cervical paravertebral musculatures at the same time.

3. An apparatus for the prevention and treatment of back pain, according to claim 1, which consists of two metal structures, one of these vertical and with three vertebral supports, these being height-adjustable and with compression springs (4); and the other horizontal, incorporating a seat (5), a traction spring (6) with a resistance control means (8); these two metal structures being perfectly articulated together by means of an axle or pivot (9), allow the taking of exercise appropriate for the prevention and treatment of back pain.
